(19) United States
(12) Patent Application Publication
(10) Pub. No.: US 2025/0361168 A1
(43) Pub. Date: Nov. 27, 2025

(54) COMBINATION OF SYNTHESIS SECTION AND BIOGAS PRODUCING UNIT

(71) Applicant: Topsoe A/S

(72) Inventors: Martin ØSTBERG, Tune (DK); Peter Mølgaard MORTENSEN, Roskilde (DK); Troels Juel FRIIS-CHRISTENSEN, Smørum (DK)

(73) Assignee: Topsoe A/S, Kgs. Lyngby (DK)

(21) Appl. No.: 18/874,678

(22) PCT Filed: Jun. 15, 2023
(86) PCT No.: PCT/EP2023/066150

§ 371 Date: Dec. 13, 2024

(30) Foreign Application Priority Data
Jun. 17, 2022 (EP) 22179660.0

(51) Int. Cl. C02F 11/04; B01J 19/00; B01J 19/24; C01B 3/38; C02F 3/28; C02F 103/20; C02F 103/32; C12P 7/04

(52) U.S. Cl. CPC C02F 11/04; B01J 19/0013; B01J 19/245; C01B 3/384; C02F 3/2866; C12P 7/04; B01J 2219/00103; B01J 2219/0011; B01J 2219/00132; C01B 2203/0233; C01B 2203/061; C01B 2203/062; C01B 2203/0833; C01B 2203/085; C01B 2203/1241; C02F 2103/20; C02F 2103/32; C02F 2301/046; C02F 2303/10

(57) ABSTRACT

A chemical plant and process are provided. The plant comprises a first biomass feed, a biomass digester arranged to receive the first biomass feed and provide a biogas stream, a reformer section arranged to receive at least a portion of the biogas stream and provide a first synthesis gas stream, and a first waste water stream, and a synthesis section arranged to receive a synthesis gas stream from the reformer section and provide a raw product stream. At least a portion of the first waste water stream is arranged to be fed to the biomass digester. In such a matter, water and heat produced downstream in the plant/process is recycled upstream in the plant/process.

### TECHNICAL FIELD

The present invention relates to a chemical plant and process for effective use of biogas, in which purification of waste water streams can be reduced or avoided.

### BACKGROUND

Biogas is a renewable energy source that can be used for heating, electricity, and many other operations. Biogas can be cleaned and upgraded to natural gas standards, to become bio-methane. Biogas is considered to be a renewable resource because its production-and-use cycle is continuous, and it generates no net carbon dioxide. When the organic material has grown, it is converted and used. It then regrows in a continually repeating cycle. From a carbon perspective, as much carbon dioxide is absorbed from the atmosphere in the growth of the primary bio-resource as is released, when the material is ultimately converted to energy.

Biogas is a mixture of gases produced by the breakdown of organic matter in the absence of oxygen. Biogas can be produced from raw materials such as agricultural waste, manure, municipal waste, plant material, sewage, green waste or food waste. Biogas is primarily methane (CH₄) and carbon dioxide (CO₂) and may include small amounts of hydrogen sulfide (H₂S), moisture, siloxanes, and possibly other components.

A biogas contains typically about 50-60% methane and 40-50% CO₂. To utilize the CO₂ in the biogas, it is advantageous to produce a syngas that can be fed to a downstream synthesis that takes advantage of the H₂/CO ratio that can be obtained. One such synthesis is a methanol (MeOH) synthesis where methanol is produced from the synthesis gas in a methanol loop. Alternatively, FT synthesis, a gasoline (TIGAS) synthesis or an acetic acid synthesis could be used.

A process and plant for converting biogas to methanol is described in WO2020254121. Waste water streams from chemical plants (e.g. methanol plants) typically comprise small amounts of impurities, e.g. organic material such as hydrocarbons, inorganic salts and dissolved gases such as CO₂. These impurities typically have to be removed before the waste water streams can be disposed of, or used as feed in other reactors in the plant, e.g. reformer reactors. In particular, small amounts of inorganic salts in a steam system of a chemical plant may build up and require additional cleaning in the plant. Also, it might be useful to use any organic materials such as hydrocarbons present in such waste water streams, thus increasing the carbon efficiency of a chemical plant. Furthermore, recycling of various streams from the chemical plant can assist in providing heat to the digester.

A high flow of water is needed for the biomass digester. Additionally, controlling the content of inorganic salts is critical for the bacteria and therefore it is necessary to have a purification of the water, removing the inorganic salts. Another important source for the biogas unit is heat. The digester operates at an optimal temperature of about 50° C., making heat supply to the digester a major cost for a plant in which biogas production takes place.

It would be desirable to provide chemical plants for effective use of biogas, in which purification of waste water streams can be reduced or avoided, in particular, allowing the waste water streams (and heat thereof) to be used elsewhere in the plant.

### SUMMARY

It has been found by the present inventor(s) that, by recycling the water rich streams from a synthesis section (e.g. a methanol loop) to the biogas unit, clean-up by e.g. stripping of these streams can be avoided, and the complexity and cost of the methanol plant can thereby be reduced.

So, in a first aspect the present invention relates to a chemical plant, said plant comprising:
a first biomass feed,
a biomass digester, arranged to receive the first biomass feed and provide a biogas stream,
a reformer section arranged to receive at least a portion of the biogas stream and provide a first synthesis gas stream, and a first waste water stream,
a synthesis section, arranged to receive a synthesis gas stream from the reformer section and provide a raw product stream;
wherein at least a portion of said first waste water stream is arranged to be fed to the biomass digester.

A process is also described for providing a raw product stream from a first biomass feed, in a chemical plant as described herein, said process comprising the steps of:
feeding the first biomass feed to the biomass digester to provide a biogas stream,
feeding at least a portion of the biogas stream from the biomass digester to the reformer section, to provide a first synthesis gas stream and a first waste water stream,
feeding at least the synthesis gas stream from the reformer section to the synthesis section, to provide a raw product stream;
feeding at least a portion of said first waste water stream to the biomass digester.

Further details of the technology are provided in the enclosed dependent claims, figures and examples.

### LEGENDS TO THE FIGURES

The technology is illustrated by means of the following schematic illustrations, in which:

FIG. 1 shows a schematic process layout of the plant of the invention.

FIG. 2 shows a schematic process layout for a plant, using electrical reforming of biogas to produce methanol.

FIG. 3 shows a schematic process layout of a distillation section suitable for upgrading the raw product stream.

### DETAILED DISCLOSURE

Unless otherwise specified, any given percentages for gas content are % by volume. All feeds are preheated as required.

The term “synthesis gas” is meant to denote a gas comprising hydrogen, carbon monoxide and also carbon dioxide and small amounts of other gasses, such as argon, nitrogen, methane, etc.

In the following a “waste water” stream is a stream comprising a majority (i.e. more than 50% by volume) of water. The waste water stream(s) may be liquid or gaseous streams, but are—in a preferred embodiment—gaseous.

A chemical plant is thus provided, which converts biomass feed to a raw product stream. In general terms, the plant comprises:
a first biomass feed,
a biomass digester,
a reformer section
and a synthesis section.

These components, their arrangement and their function will be discussed in detail in the following.

### Biomass Feed

A biomass feed is typically a liquid slurry, with a total solids content of between 20-40%. Apart from water, biomass principally comprises organic material which can be converted by the action of microbes to a biogas, e.g. in an anaerobic digestion with anaerobic organisms or methanogen inside an anaerobic digester. Sources of biomass feed include agricultural waste, such as manure, sewage, green waste and food waste, as well as industrial waste e.g. from food or drink production.

Apparatus for handling and supply of the biomass feed to the plant are known to the skilled engineer.

### Biomass Digester

A biomass digester is arranged to receive the first biomass feed and provide a biogas stream. The term “biogas” in connection with the present invention denotes a gas with the following composition:

| Compound | % |
|---|---|
| Methane | 50-75 |
| Carbon dioxide | 25-50 |
| Nitrogen | 0-10 |
| Hydrogen | 0-1 |
| Oxygen | 0-1 |

The bacteria which convert the biomass feed into biogas are capable of digesting most hydrocarbon feedstocks. This is important in the combination of a biogas unit with a chemical synthesis unit.

A biomass digester is typically in the form of a pressure reaction vessel with appropriate inlet(s) for biomass and outlet(s) for biogas. Additional inlets and outlets may be provided for the various waste water streams recycled according to the invention. Inlets and outlets may also be provided for e.g. sampling the contents of the digester or introducing or removing microbial matter.

The biomass digester operates most effectively at around 50° C. In one aspect, the plant comprises means for heating the biomass digester, preferably a heat exchanger.

Compared to a non-heated biomass digester, a heated biomass digester provides a lower residence time in the vessel, and therefore a high production.

Direct heating with steam has the disadvantage of requiring an elaborate steam-generating system (including desalination and ion exchange as water pre-treatment) and can also cause local overheating. The high cost may only be justifiable for large-scale sewage treatment facilities. The injection of hot water raises the water content of the slurry and should only be practiced if such dilution is necessary.

Indirect heating is accomplished with heat exchangers located either inside or outside of the digester, depending on the shape of the vessel, the type of substrate used, and the nature of the operating mode.
1. Floor heating systems have not served well in the past, because the accumulation of sediment gradually hampers the transfer of heat.
2. In-vessel heat exchangers are a good solution from the standpoint of heat transfer as long as they are able to withstand the mechanical stress caused by the mixer, circulating pump, etc. The larger the heat-exchange surface, the more uniformly heat distribution can be effected which is better for the biological process.
3. On-vessel heat exchangers with the heat conductors located in or on the vessel walls are inferior to in-vessel-exchangers as far as heat-transfer efficiency is concerned, since too much heat is lost to the surroundings. On the other hand, practically the entire wall area of the vessel can be used as a heat-transfer surface, and there are no obstructions in the vessel to impede the flow of slurry.
4. Ex-vessel heat exchangers offer the advantage of easy access for cleaning and maintenance.

Further components and design of the biomass digester are known to the skilled engineer.

### Reformer Section

A reformer section is arranged to receive at least a portion of the biogas stream and provide a first synthesis gas stream, and a first waste water stream.

The first synthesis gas stream typically comprises (in % by volume)
0.5-5% methane (dry)
40-70% H₂ (dry)
10-30% CO (dry)
2-20% CO₂ (dry)

This first waste water stream from the reformer section (also called a “condensate”) results from condensation of gaseous components (including water) in the reformer section, after the production of the synthesis gas.

The first waste water stream from the reformer section typically comprises (in % by volume)
99-100% H₂O
0-0.10% CO₂
<0.01% CH4,
traces of H2, CO. NH3

The first waste water stream comprises trace amounts of minerals, particularly Ni, Fe and Al salts.

At least a portion of the first waste water stream is arranged to be fed to the biomass digester, typically in admixture with the biomass feed. The first waste water stream is therefore fed into the digester vessel. Any hydrocarbons present in the first waste water stream can be digested by the microbes in the biomass digester, increasing the carbon utility of the plant. Additionally, minerals and other such trace components in the first waste water stream can function as nutrients for the microbes in the biomass digester. A content of minerals which is too high (determined by conductivity of the water, and should preferably be below 25 microsiemens/cm), can affect growth and survival of microbes.

The first waste water stream typically has an elevated temperature (e.g. between 15-60° C.) as it leaves the reformer section. The elevated temperature of this waste water stream may be advantageously used to heat the biomass digester. Heating of the biomass digester may take place via direct addition of the waste water stream to the digester vessel, i.e. by admixture with the biomass feed. Alternatively, or additionally, heating by means of the first waste water stream may take place by passing it through one or more heat exchangers located either inside or outside of the digester, as described above.

The reformer section may comprise one or more of an autothermal reforming (ATR) unit, a steam methane reforming (SMR) unit and an electrically heated steam methane reforming (e-SMR) unit, and is preferably an electrically heated steam methane reforming (e-SMR) unit. Details of an e-SMR unit that is preferably used in the reformer section are found in WO2020254121.

Additional feeds (e.g. a steam feed or oxygen-rich feed) are supplied to the reformer section, as required, depending on the type of reforming to be carried out. For instance, SMR requires a steam feed, while ATR requires a steam feed and an oxygen-rich feed.

### Synthesis Section

The synthesis section is arranged to receive a synthesis gas stream from the reformer section and provide a raw product stream.

In one preferred embodiment, the synthesis section is a methanol synthesis section and the raw product stream is a raw methanol stream.

By the term “methanol synthesis section” is understood one or several reactors configured to convert synthesis gas into methanol. Such reactors can for example be a boiling water reactor, an adiabatic reactor, a condensing methanol reactor or a gas-cooled reactor. Moreover, these reactors could be many parallel reactor shells and sequential reactor shells with intermediate heat exchange and/or product condensation. It is understood that the methanol synthesis unit also contains equipment for recycling and pressurizing syngas feed to the methanol reactor(s). All constituents of the reformer feed stream are pressurized, either separately or jointly, upstream the re-forming reactor. Typically, steam is pressurized separately, whilst the other constituents of the reformer feed stream may be pressurized jointly. The pressure(s) of the constituents of the reformer feed stream is/are chosen so that the pressure within the reforming reactor lies between 5 to 100 bar, preferably between 20 and 40 bar, or preferably between 70 and 90 bar.

In this embodiment, the module

$$M = \frac{{H}_{2} - {CO}_{2}}{CO + {CO}_{2}} .$$

of the synthesis gas fed to the methanol synthesis section is in the range of 1.5 to 2.5.

In an alternative embodiment, the synthesis section is a Fischer-Tropsch (F-T) synthesis section and the raw product stream is a raw hydrocarbon stream. In this embodiment, the synthesis gas composition should have an H₂/CO ratio slightly above 2, where the exact value depends on the choice of FT catalyst.

There are at least three ways to adjust the syngas composition to match the module M or the H2/CO ratio required for a FT synthesis.
CO₂ can be removed upstream the reformer.
Additional methane could be added (if available).
Hydrogen could be added—this will typical be downstream the reformer, but could also be upstream.

### Additional Waste Water Streams

The plant described herein may comprise additional waste water streams, which—advantageously—can also be fed to the biomass digester.

In one aspect, the plant further comprises a distillation section arranged to receive at least a portion of the raw product stream (from the synthesis section) and provide at least a purified product stream and a second waste water stream. At least a portion of the second waste water stream may be arranged to be fed to the biomass digester.

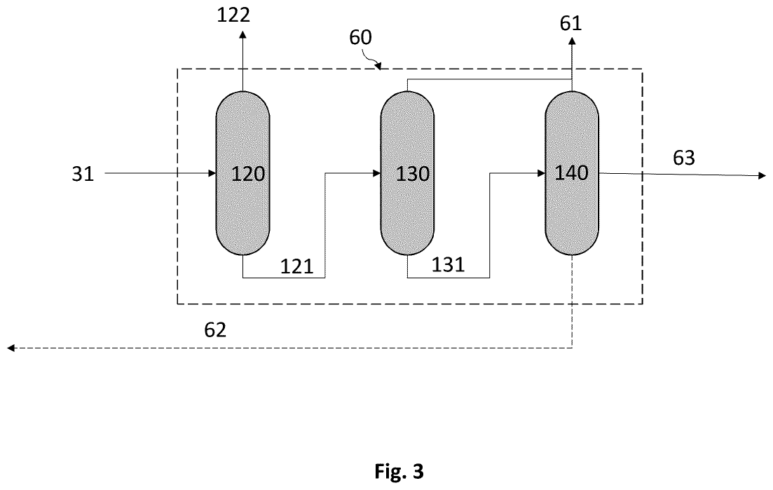

In one embodiment, illustrated in FIG. 3, the distillation section comprises a stabilizer column, a low pressure (LP) distillation column, and a medium pressure (MP) distillation column. The raw methanol product stream is purified as it passes through these columns in turn.

The MP distillation column provides a methanol stream, a waste water stream and a purge stream. The purge stream from the MP distillation column contains a significant amount of methanol, and a minor amount of higher alcohols; e.g. it typically comprises:
40-55% H₂O
40-50% CH₃OH
1-5% ethanol
0-1% 1-propanol, 2-propanol, 1-butanol, 2-butanol, 2-methyl-1-propanol, 1-pentanol
<0.01% acetone, formic acid, methyl ethyl ketone, trimethylamine.

In one embodiment, at least a portion of the purge stream from the MP distillation column is be arranged to be fed to the biomass digester.

The waste water stream from the MP distillation column typically comprises:
99.9-100% H₂O
>0.01% methanol, formic acid
Traces of ethanol, 1-propanol, 2-propanol, 1-butanol, 2-butanol, 2-methyl-1-propanol, 1-pentanol
Sodium (Na+) between 200-400 mg/l

The waste water stream from the MP distillation column may be fed to the biomass digester as the second waste water stream.

The waste water from the MP distillation column is particularly hot (ca. 150° C.). The purge stream from the MP distillation column is also particularly hot (ca. 120° C.). Usually these streams are cooled by cooling water in the plant, but in this layout the high temperatures are advantageous in heating the biomass digester. Therefore, in one aspect, the waste water stream from the MP distillation column is fed to the biomass digester, without being passed through an intermediate cooling unit. Also, in another aspect, the purge stream from the MP distillation column is fed to the biomass digester, without being passed through an intermediate cooling unit.

In another aspect, the plant may further comprise a biogas compressor arranged to compress the biogas stream from the biomass digester prior to it being fed to the reformer section. The biogas compressor is arranged to provide a third waste water stream, and at least a portion of said third waste water stream is arranged to be fed to the biomass digester. The third waste water stream from the biogas compressor typically comprises (in % by volume)
99-100% H₂O
0-0.1% CO₂
<0.01% CH₄,
traces of H₂, CO and NH₃.

In a further aspect, the plant may further comprise a syngas compressor arranged to compress the synthesis gas stream from the syngas section and provide a fourth waste water stream. At least a portion of said fourth waste water stream may be arranged to be fed to the biomass digester. The fourth waste water stream from the syngas compressor typically comprises (in % by volume)
99-100% H2O
0-0.2% CO₂
0-0.1% CO, H₂
<0.01% each of CH₄, HCN, N₂, NH₃.

In a further aspect, the plant may further comprise a steam drum arranged to produce a steam stream and a fifth waste water stream. This fifth waste water stream is commonly known as the “blow down”, and the steam drum is known as a “blow down drum”. At least a portion of the fifth waste water stream may be arranged to be fed to the biomass digester. The fifth waste water stream from the steam drum comprises almost exclusively water, e.g. 99.9-100% H₂O (% by volume). The mineral content of the fifth waste water stream is: Fe<2 mg/kg, Cu<0.3 mg/kg, silica<0.02 mg/kg, sodium<1 mg/kg, chlorine<10 mg/kg, sulphur (as sulphate)<20 mg/kg, phosphate<10 mg/kg.

These additional waste water streams are also arranged to be fed to the biomass digester, typically in admixture with the biomass feed. Any hydrocarbons present in these waste water streams can be digested by the microbes in the biomass digester, increasing the carbon utility of the plant. Additionally, minerals and other such trace components in these waste water streams can function as nutrients for the microbes in the biomass digester.

The elevated temperature of the additional (2^{nd}-5^{th}) waste water streams may be advantageously used to heat the biomass digester, directly by addition to the digester vessel or indirectly, e.g. by passing them through one or more heat exchangers located either inside or outside of the digester, as described for the first waste water stream, above.

Therefore, the plant may be arranged such that at least a portion of the first, second, third, fourth or fifth waste water streams, or a combination of two or more of said first, second, third, fourth or fifth waste water streams is arranged to be fed through a heat exchanger, thereby heating the biomass digester.

Another way in which heat can be provided to the biomass digester is by using heat energy available from one or more heat exchangers within the plant. In a particular embodiment, the reformer section and/or the synthesis section may comprise one or more heat exchangers, arranged to exchange heat between one or more cooling streams in said plant and one or more streams in the reformer section and/or the synthesis section. One or more heated streams are thus provided from the cooling streams, and at least a portion of said heated stream(s) is arranged to heat the biomass digester. The cooling streams may be cooling water streams.

### Process

The present technology also provides a process for producing a raw product stream from a first biomass feed, in a chemical plant as described herein. The process comprises the general steps of:
feeding the first biomass feed to the biomass digester to provide a biogas stream,
feeding at least a portion of the biogas stream from the biomass digester to the reformer section, to provide a first synthesis gas stream and a first waste water stream,
feeding at least the synthesis gas stream from the reformer section to the synthesis section, to provide a raw product stream;
feeding at least a portion of said first waste water stream to the biomass digester.

In one particular aspect of the process, heat is transferred from one or more of said first, second, third, fourth, and fifth waste water streams to said biomass digester. In this manner, both excess heat and waste water from the reformer section can be re-used.

In another aspect, the temperature of the biomass digester is regulated via the supply of the first waste water stream. This allows a reduction in the amount of external heat supplied to the biomass digester.

All details of the plant described above are equally relevant for the process of the invention, mutatis mutandis.

The present invention has been described with reference to a number of embodiments and figures. However, the skilled person is able to select and combine various embodiments within the scope of the invention, which is defined by the appended claims. All documents mentioned herein are incorporated by reference.

### Example 1

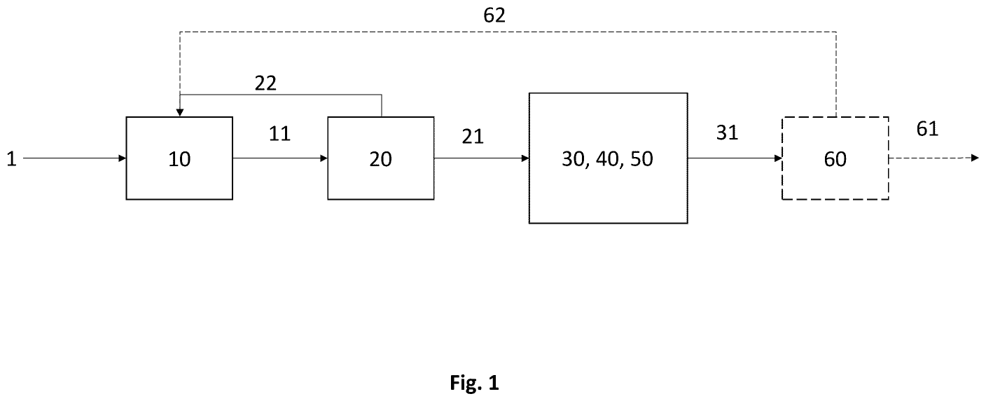

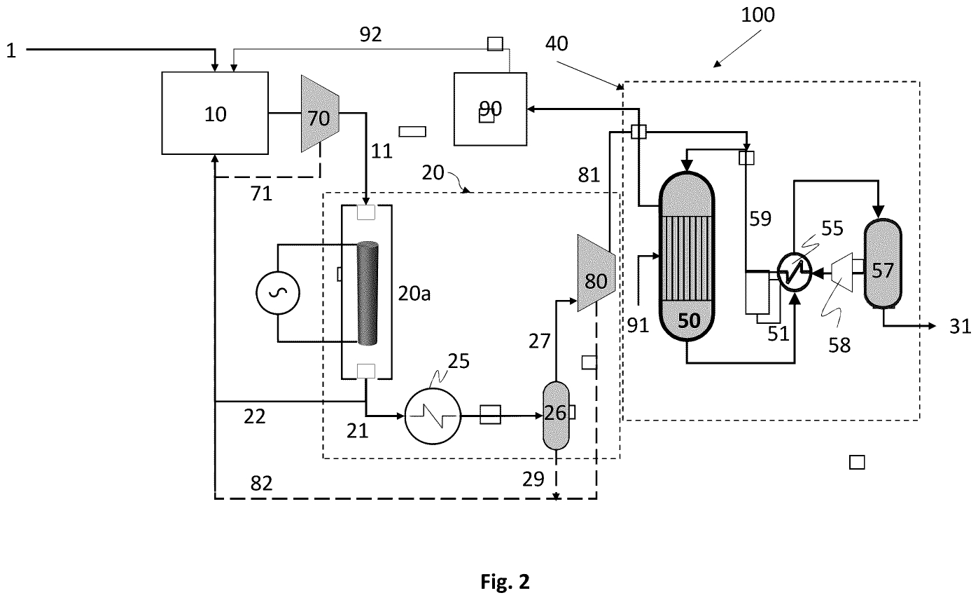

As illustrated in FIG. 1 and—in particular in FIG. 2—a methanol plant 100 is fed with biogas 11, from biomass digester 10. The biogas feed is 5000 Nm³/h biogas together with additional 193 Nm³/h biogas generated from recycled carbon. The biogas stream 11 is pretreated in a biogas upgrade unit removing a fraction of the CO₂ to meet the module for the methanol synthesis. The upgrade biogas is compressed to 25 barg and preheated to 350° C. A biogas compressor 70 collects condensed water (third waste water stream 71) and this is sent back to the biomass digester 10.

After mixing with 204 Nm³/h H₂ (recycled from the methanol unit) the biogas stream is led through a sulphur clean up unit consisting of two reactors (not shown in FIG. 2). Additional 853 Nm³/h H₂ (recycled from the methanol unit and 4158 kg/h steam is mixed with the desulphurized process stream. This process stream is heated to 450° C. and led to an adiabatic prereformer (not shown in FIG. 2). In the prereformer, the steam reforming reaction and the water gas shift reaction is equilibrated.

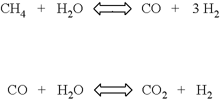

The prereformed process gas is led to the electrical heated reformer (20a), where synthesis gas stream (21) is produced. The synthesis gas leaves the reactor at 950° C. The produced synthesis gas is cooled in several heat exchangers (25) to form steam, to heat boiler feed water, to provide heat for the stabilizer column reboiler and preheat demineralised water before final cooling by cooling water to reach 40° C. The condensed water in the syngas is separated from the syngas in a separator (26) resulting in 2193 kg/h of process condensate (29).

The syngas (27) is compressed to 90 bar_{g} and mixed into the methanol synthesis loop after the recycle compressor (80). At the compression stage (80), an additional 23 kg/h process condensate (82) is formed. The mix of recycle gas and make-up syngas is preheated in a feed effluent (F/E) heat exchanger to 220° C. and led through the methanol reactor (50). The methanol reactor (50) is a boiling water reactor generating a duty of 3.69 MW thermal heat resulting in an exit temperature of 249° C. of the converted syngas (51) being cooled in the

F/E heat exchanger (55) to 114° C. Additional two heat exchangers cool the converted syngas to 40° C., which is then led to a separator (57) obtaining the condensed methanol product (31) from the synthesis section. The gaseous fraction is led to the recycle compressor (58) taking out a stream for purge and for hydrogen recycle to the front end.

After being reduced in pressure to 4 barg, the condensed methanol fraction is led to a low pressure separator (not shown) which removes a gaseous off-gas that is recycled to the biogas unit, and sends the liquid methanol fraction to a raw methanol tank in which the continuous fumes are washed with water. The water is mixed with the raw methanol in the tank, the washed gases (being another off gas stream) are sent back to the biogas unit.

Distillation section 60—FIG. 3

The raw methanol stream (31) is pumped to a stabilizer column (120) where additional off-gases (122) are evaporated from the raw methanol. The stabilized methanol stream (121) leaving the stabilizer column (120) is sent through two distillation columns (130, 140). The first distillation column (130) is at low pressure (ca. 0.8 barg) and the second (140) is at medium pressure (ca. 3.7 barg). Distilling the methanol product from species with higher boiling point also leads to a purge stream comprising higher alcohols (63) and an excess water stream (62). The excess water stream is split into a wash water stream for the raw methanol tank and a recycle stream to the biogas unit. The higher alcohol stream may also be recycled to the biogas unit.

1. A chemical plant, said plant comprising:
a first biomass feed,
a biomass digester, configured to receive the first biomass feed and provide a biogas stream,
a reformer section configured to receive at least a portion of the biogas stream, and to provide a first synthesis gas stream and a first waste water stream,
a synthesis section, configured to receive a synthesis gas stream from the reformer section and provide a raw product stream;
wherein at least a portion of said first waste water stream is configured to be fed to the biomass digester.

2. The plant according to claim 1, wherein the synthesis section is a methanol synthesis section and the raw product stream is a raw methanol stream.

3. The plant according to claim 1, wherein the synthesis section is a Fischer-Tropsch synthesis section and the raw product stream is a raw hydrocarbon stream.

4. The plant according to claim 1, further comprising a distillation section configured to receive at least a portion of the raw product stream and to provide a purified product stream and a second waste water stream, wherein at least a portion of said second waste water stream is configured to be fed to the biomass digester.

5. The plant according to claim 1, further comprising a biogas compressor configured to compress the biogas stream from the biomass digester prior to the biogas stream being fed to the reformer section, wherein said biogas compressor is configured to provide a third waste water stream, wherein at least a portion of said third waste water stream is configured to be fed to the biomass digester.

6. The plant according to claim 1, further comprising a syngas compressor configured to compress the synthesis gas stream from the syngas section and provide a fourth waste water stream, wherein at least a portion of said fourth waste water stream is configured to be fed to the biomass digester.

7. The plant according to claim 1, said plant further comprising a steam drum configured to produce a steam stream and a fifth waste water stream, wherein at least a portion of said fifth waste water stream is configured to be fed to the biomass digester.

8. The plant according to claim 1, wherein the reformer section comprises an electrically heated Steam Methane Reformer.

9. The plant according to claim 1, further comprising means for heating the biomass digester.

10. The plant according to claim 9, wherein at least a portion of said first, a second, a third, a fourth or a fifth waste water streams, or a combination of two or more of said first, second, third, fourth or fifth waste water streams, is configured to be fed through said heat exchanger, thereby heating the biomass digester.

11. The plant according to claim 1, wherein the reformer section and/or the synthesis section comprise one or more heat exchangers, configured to exchange heat between one or more cooling streams in said plant and one or more streams in said reformer section and/or said synthesis section; and thus provide one or more heated streams from said cooling streams, and wherein at least a portion of said heated stream(s) is configured to heat the biomass digester.

12. A process for producing a raw product stream from a first biomass feed, in a chemical plant according to claim 1, said process comprising the steps of:
feeding the first biomass feed to the biomass digester to provide a biogas stream,
feeding at least a portion of the biogas stream from the biomass digester to the reformer section, to provide a first synthesis gas stream and a first waste water stream,
feeding at least the synthesis gas stream from the reformer section to the synthesis section, to provide a raw product stream;
feeding at least a portion of said first waste water stream to the biomass digester.

13. The process according to claim 12, wherein heat is transferred from one or more of said first, a second, a third, a fourth or a fifth waste water streams to said biomass digester.

14. The process according to claim 12, wherein a temperature of the biomass digester is regulated by supply of the first waste water stream.

15. The plant according to claim 1, wherein the at least a portion of said first waste water stream is configured to be directly fed to the biomass digester.

16. The plant according to claim 1, wherein the at least a portion of said first waste water stream is configured to be fed to the biomass digester by admixture with the first biomass feed.
